(19) United States
(12) Patent Application Publication
(10) Pub. No.: US 2026/0272636 A1
(43) Pub. Date: Sep. 17, 2026

(54) SLEEVE FOR AN ENDOLUMINAL DEVICE HAVING AN APERTURE FOR PORTAL ACCESS OF AN IMPLANTABLE DEVICE

(71) Applicant: W. L. Gore & Associates, Inc., Newark, DE (US)

(72) Inventors: Breanna Cornell, Flagstaff, AZ (US); James A. McMaster, Newark, DE (US)

(21) Appl. No.: 19/566,677

(22) Filed: Mar. 13, 2026

Related U.S. Application Data
(60) Provisional application No. 63/772,038, filed on Mar. 14, 2025.

(51) Int. Cl. A61F 2/07; A61F 2/962

(52) U.S. Cl. CPC A61F 2/07; A61F 2/962; A61F 2240/001; A61F 2250/001

(57) ABSTRACT

An endoluminal device includes an implantable device transitionable between a collapsed, delivery configuration, an expanded, deployed configuration, and a partially deployed configuration, and a constraining sleeve removably disposed around the implantable device in the collapsed, delivery configuration. The implantable device has an intermediate portion with at least one opening in the sidewall providing access to an inner lumen of the implantable device. The constraining sleeve has an aperture aligning with the at least one opening such that the aperture provides access from an exterior to the inner lumen in the partially deployed configuration.

### CROSS-REFERENCE TO RELATED APPLICATION

This application claims the benefit of Provisional Application No. 63/772,038, filed Mar. 14, 2025, which is incorporated herein by reference in its entirety for all purposes.

### FIELD

The present disclosure relates to endoluminal devices, and more specifically, to endoluminal devices with portal access.

### BACKGROUND

There is a need for advanced devices, tools, systems and methods used for the endoluminal treatment of vascular diseases in regions of branch vessels and main vessel junctions, including treatments requiring access to branch arteries.

### SUMMARY

Endoprosthesis devices and methods of manufacturing the same are disclosed herein. In one example (“Example 1”), an endoluminal device includes an implantable device and a constraining sleeve. The implantable device is transitionable between a collapsed, delivery configuration, an expanded, deployed configuration, and a partially deployed configuration between the collapsed, delivery configuration and the expanded, deployed configuration. The implantable device has a first end portion, a second end portion, and an intermediate portion located between the first and second end portions. The intermediate portion has at least one opening in a sidewall providing access to an inner lumen of the implantable device. The constraining sleeve is removably disposed around the implantable device in the collapsed, delivery configuration. The constraining sleeve has perimeter defining an aperture aligning with at least a portion of the at least one opening with the sidewall such that the aperture provides access from an exterior to the inner lumen in the partially deployed configuration while the constraining sleeve restrains at least a portion of the implantable device in the partially deployed configuration.

In another example (“Example 2”) further to Example 1, the implantable device is self-expanding.

In another example (“Example 3”) further to Example 1 or 2, the implantable device is transitionable between the collapsed, delivery configuration and the partially deployed configuration having a greater diameter than the collapsed, delivery configuration and a smaller diameter than the expanded, deployed configuration.

In another example (“Example 4”) further to Example 3, the at least one opening defines at least one retrograde portal when the endoluminal device is disposed in an antegrade position in a vasculature such that the retrograde portal is configured to provide access in a retrograde direction with respect to the antegrade position of the endoluminal device within the vasculature.

In another example (“Example 5”) further to Example 3 or 4, the at least one opening includes at least a first opening, a second opening, and a third opening.

In another example (“Example 6”) further to Example 5, the aperture extends from a first exterior lateral edge of the first opening to a second exterior lateral edge of the second opening when the implantable device is in the partially deployed configuration.

In another example (“Example 7”) further to any one of Examples 3-6, partially deploying the constraining sleeve is operable to transition the implantable device from the collapsed, delivery configuration to the partially deployed configuration.

In another example (“Example 8”) further to any one of Examples 3-6, entirely deploying the constraining sleeve is operable to transition the implantable device from the partially deployed configuration to the expanded, deployed configuration.

In another example (“Example 9”) further to any one of Examples 1-8, the implantable device includes a stent-graft having one or more stent components and one or more graft members.

In one example (“Example 10”), a method of manufacturing an endoluminal device includes: providing an implantable device transitionable between a collapsed, delivery configuration, an expanded, deployed configuration, and a partially deployed configuration between the collapsed, delivery configuration and the expanded, deployed configuration, the implantable device having at least one opening with a sidewall providing access to an inner lumen of the implantable device; and disposing a material around the implantable device, the material defining an aperture that provides access from an exterior to the inner lumen in the collapsed, delivery configuration.

In another example (“Example 11”) further to Example 10, the aperture is formed by: removing a portion of material from the material to form the aperture; and crushing the implantable device to transition from the expanded, deployed configuration to the collapsed, delivery configuration.

In another example (“Example 12”) further to Example 10, the aperture is formed by: crushing the implantable device to transition the implantable device from the expanded, deployed configuration to the partially deployed configuration; and while the implantable device is crushed, removing a portion of material from the material that covers the at least one opening in the collapsed, delivery configuration to form the aperture.

In another example (“Example 13”) further to Example 10, the aperture is formed by: forming perforations on the material at a location associated with the at least one opening defining an outline of the aperture; crushing the implantable device to transition from the expanded, deployed configuration to the collapsed, delivery configuration; and while the implantable device is crushed, removing a portion of material from the material along the perforations to form the aperture.

In another example (“Example 14”) further to any one of Examples 10-13, the at least one opening defines at least one retrograde portal when the endoluminal device is disposed in an antegrade position in a vasculature such that the retrograde portal is configured to provide access in a retrograde direction with respect to the antegrade position of the endoluminal device within the vasculature.

In another example (“Example 15”) further to any one of Examples 10-14, the at least one opening includes at least a first opening, a second opening, and a third opening.

In another example (“Example 16”) further to Example 15, the aperture extends from a first exterior lateral edge of the first opening to a second exterior lateral edge of the second opening.

In another example (“Example 17”) further to any one of Examples 10-16, the implantable device includes a stent-graft having one or more stent components and one or more graft members.

In one example (“Example 18”), an endoluminal device includes an implantable device and a constraining sleeve. The implantable device is self-expanding and transitionable between a collapsed, delivery configuration, an intermediate deployment configuration having a greater diameter than the collapsed, delivery configuration, and an expanded, deployed configuration having a greater diameter than the collapsed, delivery configuration and the intermediate deployment configuration. The implantable device has a first end portion, a second end portion, and an intermediate portion located between the first and second end portions. The intermediate portion has a plurality of openings with a sidewall of the implantable device and providing access to an inner lumen of the implantable device. The plurality of openings including at least a first opening, a second opening, and a third opening. The constraining sleeve is removably disposed around the implantable device in the collapsed, delivery configuration and the intermediate deployment configuration, the constraining sleeve having a single aperture aligning with the plurality of openings such that the single aperture provides access from an exterior to the inner lumen in the collapsed, delivery configuration and the intermediate deployment configuration. The single aperture extends from a first exterior lateral edge of the first opening to a second exterior lateral edge of the second opening when the implantable device is in the intermediate deployment configuration. Partially deploying the constraining sleeve from the implantable device is operable to transition the implantable device from the collapsed, delivery configuration to the intermediate deployment configuration, and entirely deploying the constraining sleeve from the implantable device is operable to transition the implantable device from the intermediate deployment configuration to the expanded, deployed configuration.

In another example (“Example 19”) further to Example 18, each of the plurality of openings defines a retrograde portal when the endoluminal device is disposed in an antegrade position in a vasculature such that the retrograde portal is configured to provide access in a retrograde direction with respect to the antegrade position of the endoluminal device within the vasculature.

In another example (“Example 20”) further to Example 18 or 19, the implantable device includes a stent-graft having one or more stent components and one or more graft members.

In another example (“Example 21”), a method of treatment includes delivering an endoluminal device in a vasculature, the endoluminal device includes an implantable device, a secondary constraining sleeve disposed around the implantable device, and a primary constraining sleeve disposed around the secondary constraining sleeve such that the implantable device is in a collapsed, delivery configuration. The implantable device is partially deployed by removing the primary constraining sleeve from the secondary constraining sleeve. In a partially deployed configuration, an intermediate portion of the implantable device located between a first end portion and a second end portion of the implantable device expands through an aperture of the secondary constraining sleeve and beyond an outer surface of the secondary constraining sleeve, the intermediate portion having at least one opening in a sidewall providing access to an inner lumen of the implantable device, and the aperture aligning with at least a portion of the at least one opening with the sidewall, the aperture providing access from an exterior to the inner lumen in the partially deployed configuration while the secondary constraining sleeve restrains at least a portion of the implantable device in the partially deployed configuration. The implantable device is fully deployed by removing the secondary constraining sleeve from the implantable device. In an expanded, deployed configuration, the implantable device has a greater cross-sectional distance at the first end portion and at the second end portion than at the intermediate portion.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic diagram of an endoluminal device according to embodiments disclosed herein.

FIG. 2 is a side view of an endoluminal device in the collapsed, delivery configuration according to embodiments disclosed herein.

FIG. 3 is a side view of an endoluminal device in the expanded, deployed configuration according to embodiments disclosed herein.

FIG. 4 (prior art) is a top-down view of an endoluminal device implementing two or more sleeves as known in the art.

FIG. 5 is a flowchart of a process for manufacturing an endoluminal device according to embodiments disclosed herein.

FIGS. 6A, 6B, and 6C are cross-sectional side views of the endoluminal device in different configurations according to embodiments disclosed herein.

FIG. 7 is an angled view of the endoluminal device in a partially deployed configuration according to embodiments disclosed herein.

FIG. 8 is a side view of an endoluminal device in the expanded, deployed configuration according to embodiments disclosed herein.

### DETAILED DESCRIPTION

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

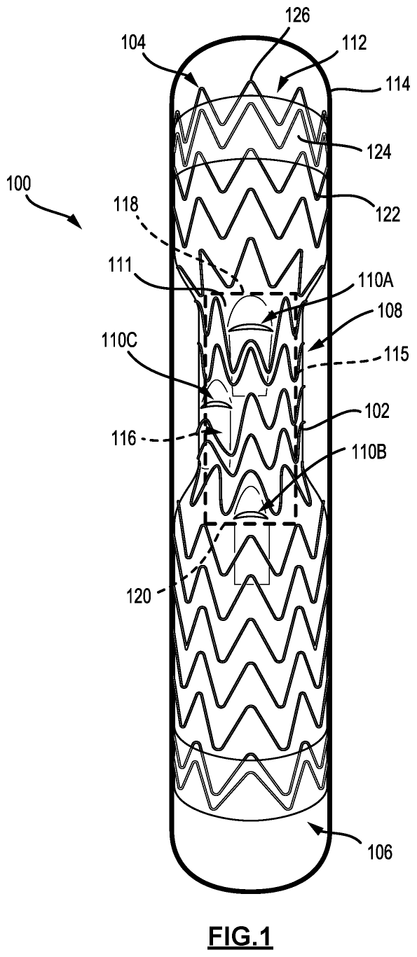

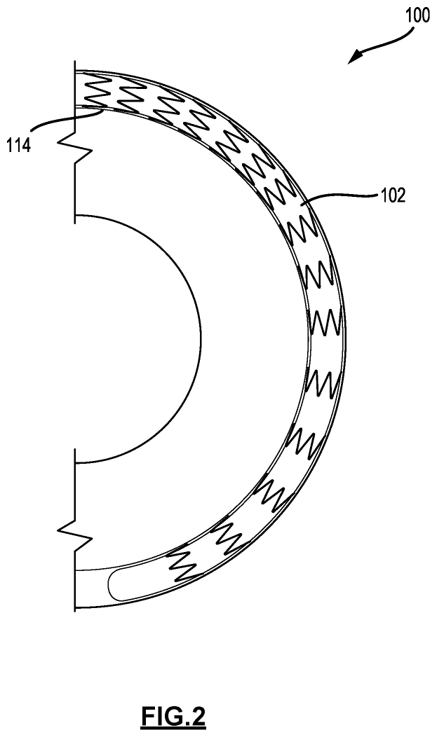

FIG. 1 shows an endoluminal device 100 according to embodiments disclosed herein. The device 100 includes an implantable device 102. In some examples, the implantable device 102 is self-expanding. In some examples, the implantable device 102 may expand with the assistance of an expanding device, such as a balloon. The implantable device 102 is transitionable between a collapsed, delivery configuration (see, for example, FIG. 2) and an expanded, deployed configuration (as shown in FIG. 1). In some examples, the implantable device 102 is also transitionable between the collapsed, delivery configuration and the partially deployed configuration having a greater diameter than the collapsed, delivery configuration, and between the partially deployed configuration and the expanded, deployed configuration. The expanded, deployed configuration has a greater diameter than the collapsed, delivery configuration and the partially deployed configuration, such that the partially deployed configuration has a smaller diameter than the expanded, deployed configuration.

The partially deployed configuration is also referred to as an intermediate deployment configuration between the collapsed, delivery configuration and the expanded, deployed configuration. The implantable device 102 has a first end portion 104, a second end portion 106, and an intermediate portion 108 located between the first end portion 104 and a second end portion 106. The intermediate portion 108 has at least one opening 110 in a sidewall 111 providing access to an inner lumen 112 of the implantable device 102. In some examples, the opening 110 includes at least a first opening 110A, a second opening 110B, and a third opening 110C. The opening 110 may also be referred to as a portal or a fenestration in the sidewall 111.

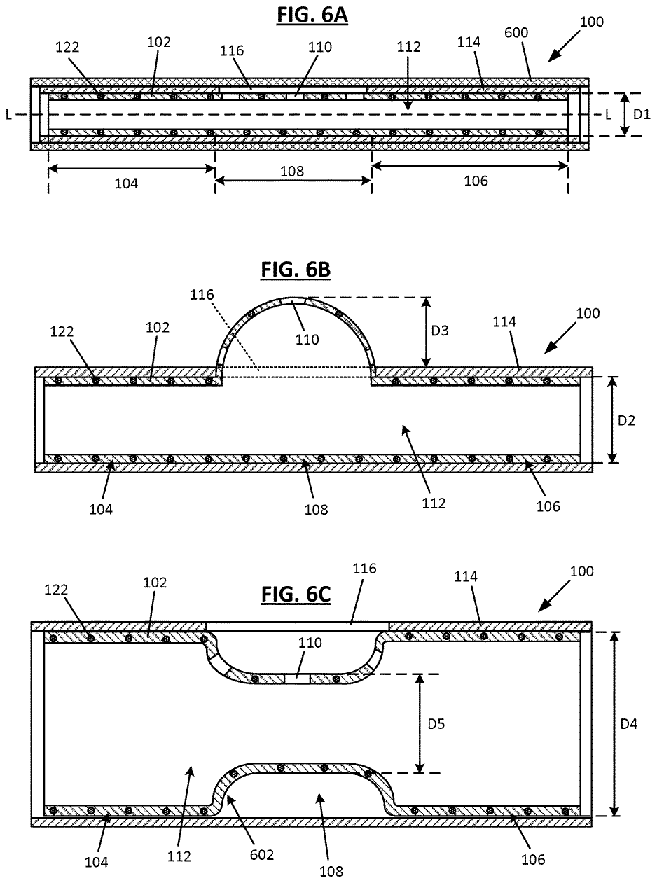

The endoluminal device 100 also includes a secondary constraining sleeve 114 that is removably disposed around the implantable device 102 in the collapsed, delivery configuration. A primary constraining sleeve (not shown in FIG. 1 but shown in FIG. 6A as a primary constraining sleeve 600) separate from the secondary constraining sleeve 114 is employed to be disposed around the secondary constraining sleeve 114 in the collapsed, delivery configuration in ordered to restrain the implantable device 102 in a constrained state with a minimal diameter for delivery. When the primary constraining sleeve is removed, the secondary constraining sleeve 114 restrains the implantable device 102 in a partially deployed configuration. In some examples, the secondary constraining sleeve 114 is removably disposed around the implantable device 102 in the partially deployed configuration. The secondary constraining sleeve 114 has a perimeter 115 that defines an aperture 116 (the perimeter 115 is shown by a dotted line in FIGS. 1, 3, and 8) aligning with the opening(s) 110 such that the aperture 116 provides access from an exterior to the inner lumen 112 in a partially deployed configuration while the secondary constraining sleeve 114 restrains at least a portion of the implantable device 102 in the partially deployed configuration.

In some examples, the aperture 116 may provide access to the inner lumen 112 in the partially deployed configuration during which the secondary constraining sleeve 114 constrains at least a portion of the implantable device 102 such that the implantable device 102 is in the partially deployed configuration. By providing the access to the inner lumen 112 in the partially deployed configuration, one or more side branch devices (such as catheters or stent-grafts) may be allowed to pass through the opening(s) while maintaining a fluid communication with the lumen 112 even while the implantable device 102 remains in the partially deployed configuration. In some examples, the secondary constraining sleeve 114 has only a single aperture 116. In some examples, the secondary constraining sleeve 114 may have a plurality of apertures 116, such as separate apertures 116 that align with one or more of the opening(s) 110.

In some examples, the aperture 116 extends from a first exterior lateral edge 118 of a first opening 110A to a second exterior lateral edge 120 of the second opening 110B when the implantable device is in the partially deployed configuration. In some examples, partially deploying the secondary constraining sleeve 114 from the implantable device 102 is operable to transition the implantable device 102, or causes the implantable device 102 to transition from the collapsed, delivery configuration to the partially deployed configuration, and entirely deploying the secondary constraining sleeve from the implantable device causes the implantable device to transition from the partially deployed configuration to the expanded, deployed configuration.

The implantable device 102 may be any suitable endoluminal devices such as a stent-graft with one or more stent components 122 and one or more graft members 124, such as with one or more graft members 124 disposed over and/or under the stent components 122, which can dilate from a delivery configuration (collapsed, delivery configuration), through a range of larger intermediary configurations (partially deployed configuration), and toward a deployed configuration (expanded, deployed configuration) engaged with vessel walls at a treatment site. The stent components 122 may have one or more apices 126 disposed along a periphery of the graft member(s) 124.

Any suitable combination and configuration of stent component(s) and graft member(s) is within the scope of the present disclosure. For example, the stent-graft may be those used in GORE® TAG® Thoracic Branch Endoprosthesis (“TBE”), GORE® VIABAHN® Balloon Expandable (“VBX”) Endoprosthesis, Conformable GORE® TAG® Thoracic Endoprosthesis (“CTAG”) or GORE® TAG® Conformable Thoracic Stent Graft with ACTIVE CONTROL System (“CMDS”), available from W. L. Gore & Associates, Inc. (Newark, DE). The sleeve may be made of any suitable an introducer sheath such as GORE® DrySeal Sheath, available from W. L. Gore & Associates, Inc.

Stent components can have various configurations such as, for example, rings, cut tubes, wound wires (or ribbons) or flat patterned sheets rolled into a tubular form. Stent components can be formed from metallic, polymeric or natural materials and can comprise conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol and biologically derived materials such as bovine arteries/veins, pericardium and collagen. Stent components can also comprise bioresorbable materials such as poly(amino acids), poly(anhydrides), poly(caprolactones), poly(lactic/glycolic acid) polymers, poly(hydroxybutyrates) and poly(orthoesters). In some examples, the stent components may be GORE® Ascending Stent Graft (“ASG”), manufactured by W. L. Gore & Associates, Inc.

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

A biocompatible material for the graft components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, an ultra high molecular weight polyethylene, a polyethylene terephthalate, fluoropolymers such as perfluoroelastomers and the like, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers. Other embodiments for a graft member material can include high strength polymer fibers such as ultra high molecular weight polyethylene fibers (e.g., Spectra®, Dyneema Purity®, etc.), aramid fibers (e.g., Technora®, etc.), and/or combinations thereof. The graft member may include a bioactive agent. In one embodiment, an ePTFE graft includes a carbon component along a blood contacting surface thereof. Any graft member which can be delivered by a catheter is in accordance with the present disclosure. Further detail of materials and general construction of stents, graft members and stent grafts are generally disclosed in U.S. Pat. Nos. 6,042,605; 6,361,637; and 6,520,986 all to Martin et al.

In FIG. 1, the openings 110 include the first opening 110A aligned with one end (first exterior lateral edge 118) of the single aperture 116, the second opening 110B aligned with another end (second exterior lateral edge 120) of the single aperture 116, and the third opening 110C located between the first opening 110A and the second opening 110B along the longitudinal direction of the device 102. In some examples, the opening(s) 110 may define at least one retrograde portal when the endoluminal device 100 is disposed in an antegrade position in a vasculature such that the retrograde portal may provide access in a retrograde direction with respect to the antegrade position of the endoluminal device 100 within the vasculature. A retrograde direction is substantially opposite from an antegrade direction providing access in an antegrade direction with respect to the antegrade position of the endoluminal device 100. In some examples, the opening(s) 110 may define at least one antegrade portal. In some examples, all openings are retrograde portals when the implantable device is positioned in an antegrade position within a vasculature. In some examples, alternatively, all openings are antegrade portals when the implantable device is positioned in the antegrade position within the vasculature. Therefore, the directions of the portals are substantially the same with respect to a longitudinal axis of the implantable device, such that each of the first opening 110A, the second opening 110B, and the third opening 110C defines a separate retrograde portal or a separate antegrade portal that is separately and independently accessible. It is to be understood that, in some examples, there may also be a combination of retrograde portal(s) and antegrade portal(s) as suitable. In some examples, the first opening 110A may be an opening that provides access of the lumen 112 of the implantable device 102 toward the direction of a brachiocephalic artery (BCA), hereinafter referred to as a BCA opening. The second opening 110B may be an opening that provides access of the lumen 112 toward the direction of a left subclavian artery (LSA), hereinafter referred to as an LSA opening. The third opening 110C may be an opening that provides access of the lumen 112 toward the direction of a left common carotid artery (LCCA), hereinafter referred to as an LCCA opening.

In the above regard, the secondary constraining sleeve 114 may be customized to align the aperture 116 with the BCA opening, LSA opening, and LCCA opening when the implantable device 102 is in the partially deployed configuration. As such, the secondary constraining sleeve 114 may be formed in a perforated state as customized depending on the locations of the openings 110 of the implantable device 102.

Methods or processes of customizing the secondary constraining sleeve 114 are explained herein. The sleeve material is provided such that a material for a sleeve (which may be in the form of a sheet material) wraps around the implantable device 102 while the implantable device 102 is in the intermediate deployment configuration. In the intermediate deployment configuration, a portion of the sleeve material is removed, such as by cutting using any suitable tool, to form an opening or aperture in the sleeve material. The portion that is removed aligns with a portion of the implantable device 102 extending from the first exterior lateral edge 118 (posterior edge) of the first opening 110A (e.g., BCA opening) to the second exterior lateral edge 120 (anterior edge) of the second opening 110B (e.g., LSA opening). In some examples, the portion that is removed aligns with a proximal edge of the BCA opening, the posterior edges of the first and second openings 110A and 110B (e.g., BCA and LSA openings), and the anterior edge of the third opening 110C (e.g., LCCA opening). The process of reducing the diameter of the implantable device 102 from the intermediate deployment configuration to the collapsed, delivery configuration, from the expanded, deployed configuration to the intermediate deployment configuration, or from the expanded, deployed configuration to the collapsed, delivery configuration such that the implantable device 102 is flushed, is referred to as “crushing.” An inner crush refers to a configuration in which the inner component (such as the implantable device 102) is reduced in diameter along at least a portion of the inner component along the longitudinal length of the inner component.

For example, before the inner crush, the secondary constraining sleeve 114 may have a portion of the sleeve material removed through cutting out the portion using a jig or any other suitable tool, in order to identify the appropriate cut locations on the secondary constraining sleeve 114 to be aligned with the openings. In some examples, subsequently, after the inner crush, the sleeve material may be removed by cutting the sleeve that is loaded on (or around) the implantable device 102. The implantable device 102 may be protected by a shield member during the cutting process so as to minimize the risk of the implantable device 102 being inadvertently cut during the process. The shield member may be any material suitable for protecting the surface or material of the implantable device, such as one or more Kapton® polyimide films provided by DuPont (Wilmington, DE).

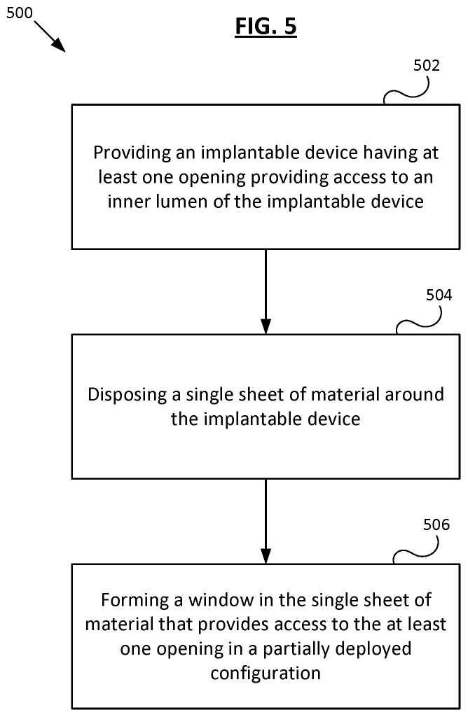

Processes of manufacturing the endoluminal device are also disclosed herein. Referring to FIG. 5, a manufacturing process 500 may be a method that includes a step 502 of providing an implantable device that has at least one opening providing access to an inner lumen of the implantable device. For example, the implantable device may have a first end portion, a second end portion, and an intermediate portion located between the first and second end portions, and the intermediate portion may have the at least one opening providing access to the inner lumen of the implantable device. The implantable device may be transitionable between a collapsed, delivery configuration and an expanded, deployed configuration. The method includes a step 504 of disposing a material (e.g., the material for the secondary constraining sleeve) around the implantable device, and, in some examples, may also include a step 506 of forming, creating, or affecting an aperture in the material that provides access to the at least one opening in the collapsed, delivery configuration. In some examples, the material may already be preformed to define an aperture. In some examples, the forming, creating, or affecting of the aperture in step 506 can be achieved at one or more of the different points during the manufacturing process 500, as explained herein using Paths 1, 2, and 3. The material may be a single sheet of material, as suitable.

In some examples (Path 1), the step 506 of forming the aperture includes a step of marking (such as via cutting) portions of the single sheet of material that align with the at least one opening. Also included is a step of removing a portion of material from the single sheet that covers the marked portions of the single sheet of material in order to form the aperture. In some examples, the marking may be optional, and the portion of material may be removed directly from the single sheet, such as via cutting the appropriate portion of material from the sheet. Subsequently, after the aperture is formed, the method includes a step of crushing (e.g., using an appropriate inner crush funnel or any other suitable tools) the implantable device to transition from the expanded, deployed configuration to the collapsed, delivery configuration. The seamline of the sleeve is then ensured to be positioned appropriately such that the aperture providing access to the opening(s) is aligned with the actual location of the opening(s), as appropriate.

In some examples (Path 2), the step 506 of forming the aperture includes a step of forming perforations on a single sheet at a location associated with the at least one opening. For example, the location may be adjacent to the opening, proximal to the opening, relative to the opening, or overlayed by the opening. In some examples, the location may be determined to be at or near the relative position of the opening, such as within a predetermined distance or within a range of distances, as suitable. For example, the predetermined distance may be the smallest distance necessary to form an aperture that exposes at least a portion of each of the opening(s) of the implantable device when the sheet is overlaid on or surrounds the implantable device. The range of distances may define the distances that may be employed to achieve the same; for example, the minimal distance may be the smallest distance necessary to form an aperture to expose at least 50% (or alternatively, 30%, 40%, 60%, 70%, or any other suitable value between these ranges) of the area of each opening, and the maximal distance may be the smallest distance necessary to form an aperture to expose up to 100% of the area of each opening, such that the range of distances is between the minimal distance and the maximal distance. The perforations may define an outline of the aperture. Subsequently, a step of crushing (by using the appropriate tool such as the inner crush funnel) the implantable device to transition from the expanded, deployed configuration to the collapsed, delivery configuration is included. The seamline of the sleeve is then ensured to be positioned appropriately such that the perforations are aligned with the actual location of the opening(s), as appropriate. In some examples, while the implantable device is crushed, the step of forming the aperture includes a step of removing a portion of material from the single sheet along the perforations formed on the single sheet (e.g., the perforations may be preformed as part of the customization at the location for material removal), in order to form the aperture and thus exposing the opening(s).

In some examples (Path 3), the step 506 of forming the aperture includes a step of crushing (by using the appropriate tool such as the inner crush funnel) the implantable device to transition from the expanded, deployed configuration to the collapsed, delivery configuration. The seamline of the sleeve is then ensured to be positioned appropriately. The step of forming the aperture also includes a step of removing (e.g., using any suitable cutting tool such as scissors) a portion of material (e.g., in one piece) from the single sheet that covers the at least one opening in the collapsed, delivery configuration while the implantable device is crushed, in order to form the aperture. The removal of the portion of material is performed without damaging the implantable device or the opening(s).

In some examples, the method further includes a step of disposing a shield material around the implantable device before the single sheet of material is disposed around the implantable device (e.g., between steps 502 and 504 in FIG. 5) such that the shield material protects the implantable device during the removing of the portion of material from the single sheet, for example by cutting the single sheet of material. In some examples, the at least one opening include at least a first opening, a second opening, and a third opening. The first opening may provide access of the lumen toward a direction of the BCA (i.e., a BCA opening), the second opening may provide access of the lumen toward a direction of the LSA (i.e., an LSA opening), and the third opening may provide access of the lumen toward a direction of the LCCA (i.e., an LCCA opening). In some examples, the aperture is formed such that the aperture extends from a first exterior lateral edge of the first opening to a second exterior lateral edge of the second opening. In some examples, the implantable device may include a stent-graft having one or more stent components and one or more graft members. It is to be understood that, although a single sheet of material is mentioned, additional variations of the material may be implemented, such as, for example, a single membrane, a plurality of sheets, a plurality of membranes, or a combination of sheets and membranes.

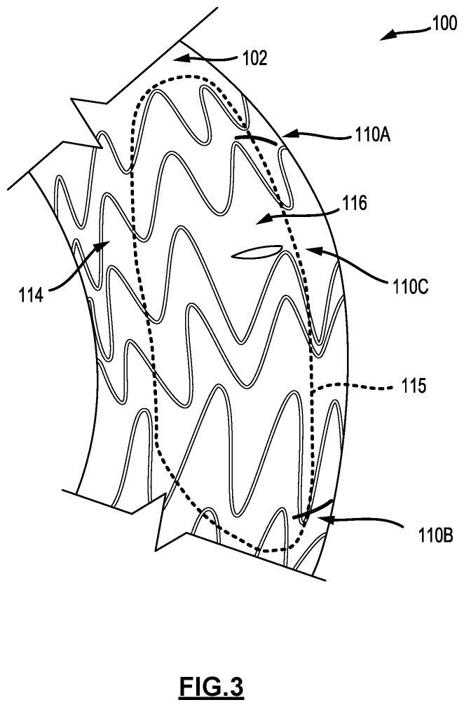

FIG. 2 shows the endoluminal device 100 when the implantable device 102 and the secondary constraining sleeve 114 are in a reduced diameter, such as the collapsed, delivery configuration, in a simulated vasculature. The secondary constraining sleeve 114 maintains the implantable device 102 in the collapsed, delivery configuration. When the secondary constraining sleeve 114 is released, the implantable device 102 is allowed to deploy to transition to the partially deployed configuration, such as by self-expanding. In FIG. 3, the aperture 116 is visibly more open, thus providing access for the openings 110A, 110B, and 110C which are also visible. Access to the openings is maintained while the secondary constraining sleeve 114 constrains the implantable device 102 at the partially deployed configuration.

FIG. 6A shows an example of the endoluminal device 100 in which the implantable device 102 is constrained in the collapsed, delivery configuration using both a primary constraining sleeve 600 and the secondary constraining sleeve 114 disposed within the primary constraining sleeve 600 and external to the implantable device 102. The implantable device 102, the primary constraining sleeve 600, and the secondary constraining sleeve 114 are coaxial with respect to a longitudinal axis L-L. All cross-sectional distances are measured along a plane perpendicular to the longitudinal axis. If the cross-sectional shape is substantially circular, the cross-sectional distance may also be referred to as a diameter. In the collapsed, delivery configuration, the implantable device 102 defines a cross-sectional distance D1 throughout the longitudinal length so as to have consistent cross-sectional distance D1 at the first end portion 104, the second end portion 106, and the intermediate portion 108. The aperture 116 of the secondary constraining sleeve 114 extends across all openings 110 of the implantable device 102, and the aperture 116 is covered by the primary constraining sleeve 600.

FIG. 6B shows the endoluminal device 100 in which the primary constraining sleeve 600 is removed or retracted such that the intermediate portion 108 of the implantable device 102 is allowed to expand beyond an outer surface of the secondary constraining sleeve 114 as the secondary constraining sleeve 114 still operates to constrain the first end portion 104 and the second end portion 106 of the implantable device 102 but allowing the intermediate portion 108 to expand to have a greater maximal cross-sectional distance than either of the first end portion 104 and the second end portion 106, so as to assume the partially deployed configuration of the implantable device 102. In the partially deployed configuration, the implantable device 102 defines a cross-sectional distance D2 at the first end portion 104 and the second end portion 106, while the intermediate portion 108 has a maximal cross-sectional distance of D2+D3, where D2 may be greater than D1 (D2>D1) and D3 is the distance by which the farthest portion of the intermediate portion 108 extends past the outer surface of the secondary constraining sleeve 114 by protruding outwardly from the aperture 116 of the secondary constraining sleeve 114.

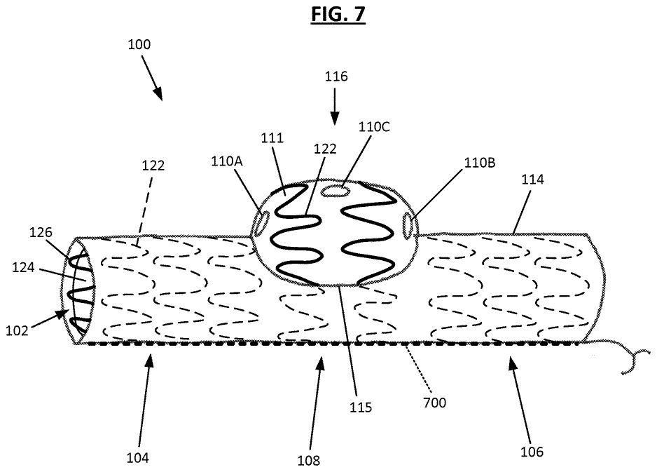

FIG. 6C shows the endoluminal device 100 in which the implantable device 102 is in the expanded, deployed configuration, or a fully deployed configuration. This is achieved by removing or releasing the secondary constraining sleeve 114 such as by retracting the sleeve or by allowing the sleeve to be released from the implantable device 102 such that the secondary constraining sleeve 114 no longer applies any constraining force on the implantable device 102. For example, if the secondary constraining sleeve 114 is formed using a single sheet of material with two opposing edges that are releasably coupled or tied together (such as via the use of a release line 700 as shown in FIG. 7, for example), the two opposing edges of the secondary constraining sleeve 114 may be released from the coupling by retracting the release line 700 to release the secondary constraining sleeve 114 from the implantable device 102.

Once released and unconstrained, the implantable device 102 assumes the expanded, deployed configuration in which the first end portion 104 and the second end portion 106 of the implantable device 102 has a greater cross-sectional distance D4 than the cross-sectional distance D5 at the intermediate portion 108 (D4>D5). In some examples, the difference between the distances D4 and D5 may cause the implantable device 102 to have one or more tapered portions 602 formed between the intermediate portion 108 and at least one of the first end portion 104 or the second end portion 106. In some examples, D5 is greater than the sum of D2 and D3 (D5>D2+D3), or D5 may be substantially equal to the sum of D2 and D3 (D5=D2+D3). When there are two tapered portions 602 (e.g., a first tapered portion between the intermediate portion 108 and the first end portion 104 and a second tapered portion between the intermediate portion 108 and the second end portion 106), the implantable device 102 may be referred to as having an hourglass or dumbbell configuration. In some examples, the tapered portion(s) 602 may provide additional room for a physician to use during operations such as when performing medical procedures including but not limited to cannulation, for example.

FIG. 7 shows an example of the endoluminal device 100 in which the implantable device 102 assumes the partially deployed configuration. In some examples, the secondary constraining sleeve 114 extends beyond the apices 126 of the stent components 122 such that all of the apices 126 are covered by or encapsulated within the secondary constraining sleeve 114 when the secondary constraining sleeve 114 is applying the constraining force on the implantable device 102. This is to prevent having any un-taped apices (e.g., any of the apices 126 not taped or attached to a corresponding graft member 124) presenting a possible risk of having an edge of the sleeves (e.g., the secondary constraining sleeve 114 or the primary constraining sleeve 600) being caught by a tip of the un-taped apex.

The intermediate portion 108 may be allowed to expand such that a portion of the stent components 122 (shown in bold solid lines) and the corresponding graft member(s) 124 that define the sidewall 111 are exposed along with the one or more openings 110 (e.g., 110A, 110B, and 110C as shown) by protruding outwardly from the aperture 116 defined by the perimeter 115 as formed in the secondary constraining sleeve 114, whereas the remaining portion of the stent components 122 (shown in broken lines) and the graft member(s) 124 of the implantable device 102 remain constrained within the secondary constraining sleeve 114. In some examples, when there are a plurality of openings 110, each of the openings 110 may be facing a different direction from the other openings 110 depending on a curvature in the sidewall 111 near the openings 110.

In some examples, in the partially deployed configuration, the stent components 122 may self-expand (such as when the stent components 122 are formed using a self-expanding material such as a shape memory alloy) to protrude outwardly from the aperture 116. In some examples, the stent component 122 may expand at a greater degree than the graft member(s) 124 defining the sidewall 111. In some examples, the graft member(s) 124 may expand (such as by using an inflatable balloon that is inflated within the implantable device 102) and exert an outward force on the stent component 122 to cause expansion of the intermediate portion 108 of the implantable device 102.

The secondary constraining sleeve 114 may implement at least one release line 700 (shown in a bold dotted line) to control the operation of the secondary constraining sleeve 114 with respect to the implantable device 102. Upon release, the release line 700 allows the entirety of the implantable device 102 to fully expand to transition from the partially deployed configuration to the expanded, deployed configuration. More than one release line may be implemented to flexibly control the deployment process of the implantable device 102. The release line 700 may be made of any suitable material (such as a string, fiber, or polymer such as those that may be used to form the graft member 124) capable of releasably coupling together the opposing edges of the secondary constraining sleeve 114 (such as by temporarily sewing the edges together until the edges are released by unraveling the sewn portion). The release line 700 is located away from the aperture 116 such that the release line 700 does not affect the functionality of the aperture 116, and vice versa.

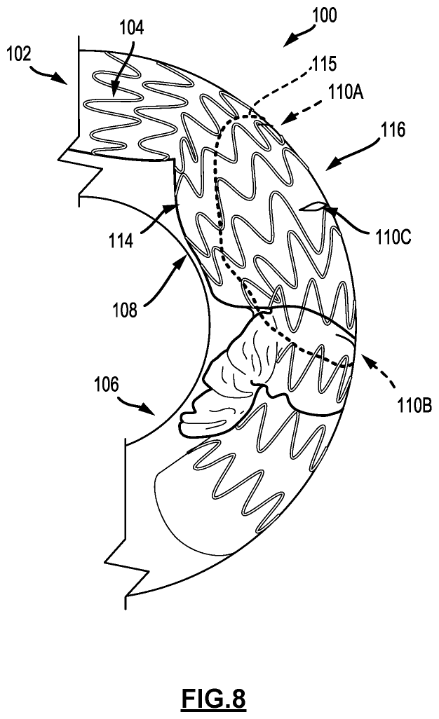

FIG. 8 shows an example of the endoluminal device 100 in which the implantable device 102 assumes the partially deployed configuration within a simulated vasculature. The aperture 116 allows the openings 110 to be accessible during the partially deployed configuration (the opening 110C is visible whereas the other openings 110A and 110B are hidden from view in the angle as shown), but because the intermediate portion 108 of the implantable device 102 is in contact with the wall of the simulated vasculature, the intermediate portion 108 cannot expand outwardly from the aperture 116 of the secondary constraining sleeve 114. Instead, the side of the secondary constraining sleeve 114 at the intermediate portion 108 that is opposite from where the aperture 116 is located is allowed to expand against the secondary constraining sleeve 114, which forces the aperture 116 to widen in order to accommodate the expansion of the implantable device 102 at the intermediate portion 108. As such, as shown, the aperture 116 (shown in a dotted line) is opened substantially wider but the intermediate portion 108 may not necessarily protrude outwardly from the aperture 116 when the expansion is hindered by the vasculature wall.

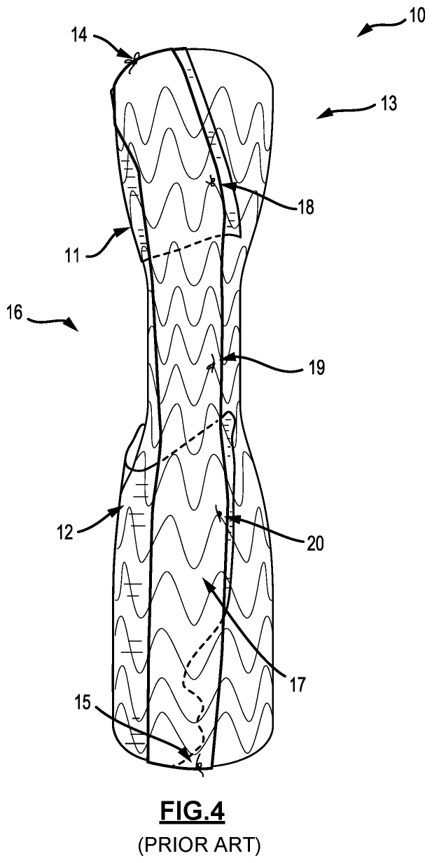

Currently, prior-art endoluminal devices as known in the art require two or more separate sleeves that separately facilitate the crushing of an implantable device. For example, as shown in FIG. 4, a prior-art endoluminal device 10 is provided with a proximal sleeve 11 and a distal sleeve 12 such that one end portion (proximal end portion 14) of the implantable device 13 is constrained using the proximal sleeve 11, and the other end portion (distal end portion 15) of the implantable device 13 is constrained using the distal sleeve 12, leaving a middle portion 16 that is not entirely covered by either of the two (or more, in some examples) sleeves 11 and 12 to provide access to any openings (not shown) that are formed in the implantable device 13 that provide access to its inner lumen within the implantable device 13. Also shown is an outer sleeve 17 positioned over the proximal sleeve 11 and the distal sleeve 12. Attachments 18, 19, and 20 are also provided to attach the sleeves to the implantable device 13. Attachments are also be provided at the end portions 14 and 15.

However, using two or more separate sleeves to facilitate the crushing of the implantable device introduces uncertainties to the endoluminal device that are not immediately noticeable due to the location of the proximal sleeve being in a region of the device that is different from that of the predicate devices. One such observed issue is that a line or a tether that is used for deployment of the device can be inadvertently captured at an un-taped apex tip of one of the stent components during deployment. Another issue is that, if an apex portion of one of the stent components is bent backward and is not covered with any sleeve, the backwardly-bent portion can be caught in a funnel during or after deployment. Addressing such uncertainties as they are learned leads to increased manufacturing complexity.

Furthermore, in prior-art devices, the customization process of two or more sleeves and then routing the deployment lines would increase the time required to do so, as compared to the predicate devices with similar processes such as CMDS and ASG, both of which are available from or manufactured by W. L. Gore & Associates, Inc. In some cases, the customization of two or more sleeves and the routing of two or more deployment lines also increase the ergonomic impact for these steps as compared to predicate devices with similar processes. In some cases, the customization, the crushing, and the routing (or fiber management) of two or more deployment lines introduces additional amount of potential impacts to yield, thereby allowing errors to occur during the manufacturing process. In some cases, the use of two or more sleeves increases the material cost and material waste associated with the sleeves as compared to the predicate products with similar processes. In some cases, un-taped apices, or the apices of the stent components that are not covered by the sleeves, may present additional risks, such as the edge of the sleeves being caught by a tip of the un-taped apex.

Risks of such problems as listed above can be minimized or reduced using the secondary constraining sleeve 114 with the aperture 116 for constraining the implantable device 102 as disclosed herein. For example, the risk of crushing the implantable device 102 into two or more sleeves with yet unknown uncertainties (e.g., uncertainties related to the sleeves' seamlines) can be addressed by aligning the aperture 116 with the openings 110. In manufacturing, the takt time (that is, the required product assembly duration needed to match the demand for the product) of the endoluminal device 100 can be reduced to be comparable to the predicate devices (such as CMDS and ASG). Also, the ergonomic impacts of sleeve customization can remain consistent with the aforementioned predicate devices. The anticipated yield impacts can also remain consistent with the aforementioned predicate devices. The amount of material cost and material waste can also remain consistent with the aforementioned predicate devices. Furthermore, using the secondary constraining sleeve 114 to cover the un-taped apex region of the implantable device 102 (that is, the apices 126 of the stent components 122) allows multibranch arch (“MBA”) graft devices to be aligned with the aforementioned predicate devices, thereby allowing for alignment on future improvements. Additionally, doing so removes the risk of an edge of any additional outer sleeve to be caught on an un-taped apex tip of the stent components 122 while the additional outer sleeve moves away from the opening region during deployment. Such risk is not only minimized for the MBA devices using the embodiments disclosed herein, but also for future improvements to the attachment locations for the additional outer sleeve.

Furthermore, the endoluminal device as disclosed herein facilitates simple and precise delivery of a multi-branch endograft through staged deployment, as well as facilitating passive rotational alignment and femoral branch delivery, and also providing reliable opening access through rail guides. The device also provides durable arch treatment and has broad clinical applicability with retrograde openings that facilitate broad anatomical fit, such as for thoracic aneurysm, chronic dissections, anastomotic pseudoaneurysm after previous surgical repair of the thoracic aorta or aortic heart valve, and penetrating atherosclerotic aortic ulcer (PAU) with or without aortic intramural hematoma (IMH). The device may be implemented with other medical treatment products and devices such as ASG, MBA devices, TBE, VBX endoprosthesis, and CTAG. The device facilitates minimizing or reducing the risk of stroke by reducing manipulation via passive rotational alignment and simple opening access, providing retrograde branch delivery, and providing compatibility with embolic protection system (EPS).

It is further understood that sleeves such as the secondary constraining sleeve may be implemented not only as a secondary or second sleeve, but may be implemented as the only sleeve constraining a device or may be used with additional sleeves beyond what is described herein.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An endoluminal device comprising:
an implantable device transitionable between a collapsed, delivery configuration, an expanded, deployed configuration, and a partially deployed configuration between the collapsed, delivery configuration and the expanded, deployed configuration, the implantable device having a first end portion, a second end portion, and an intermediate portion located between the first and second end portions, the intermediate portion having at least one opening in a sidewall providing access to an inner lumen of the implantable device; and
a constraining sleeve removably disposed around the implantable device in the collapsed, delivery configuration, the constraining sleeve having perimeter defining an aperture aligning with at least a portion of the at least one opening with the sidewall such that the aperture provides access from an exterior to the inner lumen in the partially deployed configuration while the constraining sleeve restrains at least a portion of the implantable device in the partially deployed configuration.

2. The endoluminal device of claim 1, wherein the implantable device is self-expanding.

3. The endoluminal device of claim 1, wherein the implantable device is transitionable between the collapsed, delivery configuration and the partially deployed configuration having a greater diameter than the collapsed, delivery configuration and a smaller diameter than the expanded, deployed configuration.

4. The endoluminal device of claim 3, wherein the at least one opening defines at least one retrograde portal when the endoluminal device is disposed in an antegrade position in a vasculature such that the retrograde portal is configured to provide access in a retrograde direction with respect to the antegrade position of the endoluminal device within the vasculature.

5. The endoluminal device of claim 3, wherein the at least one opening includes at least a first opening, a second opening, and a third opening.

6. The endoluminal device of claim 5, wherein the aperture extends from a first exterior lateral edge of the first opening to a second exterior lateral edge of the second opening when the implantable device is in the partially deployed configuration.

7. The endoluminal device of claim 3, wherein partially deploying the constraining sleeve is operable to transition the implantable device from the collapsed, delivery configuration to the partially deployed configuration.

8. The endoluminal device of claim 3, wherein entirely deploying the constraining sleeve is operable to transition the implantable device from the partially deployed configuration to the expanded, deployed configuration.

9. The endoluminal device of claim 1, wherein the implantable device comprises a stent-graft having one or more stent components and one or more graft members.

10. A method of manufacturing an endoluminal device, the method comprising:
providing an implantable device transitionable between a collapsed, delivery configuration, an expanded, deployed configuration, and a partially deployed configuration between the collapsed, delivery configuration and the expanded, deployed configuration, the implantable device having at least one opening with a sidewall providing access to an inner lumen of the implantable device; and
disposing a material around the implantable device, the material defining an aperture that provides access from an exterior to the inner lumen in the collapsed, delivery configuration.

11. The method of claim 10, wherein the aperture is formed by:
removing a portion of material from the material to form the aperture; and
crushing the implantable device to transition from the expanded, deployed configuration to the collapsed, delivery configuration.

12. The method of claim 10, wherein the aperture is formed by:
crushing the implantable device to transition the implantable device from the expanded, deployed configuration to the partially deployed configuration; and
while the implantable device is crushed, removing a portion of material from the material that covers the at least one opening in the collapsed, delivery configuration to form the aperture.

13. The method of claim 10, wherein the aperture is formed by:
forming perforations on the material at a location associated with the at least one opening defining an outline of the aperture;
crushing the implantable device to transition from the expanded, deployed configuration to the collapsed, delivery configuration; and
while the implantable device is crushed, removing a portion of material from the material along the perforations to form the aperture.

14. The method of claim 10, wherein the at least one opening defines at least one retrograde portal when the endoluminal device is disposed in an antegrade position in a vasculature such that the retrograde portal is configured to provide access in a retrograde direction with respect to the antegrade position of the endoluminal device within the vasculature.

15. The method of claim 10, wherein the at least one opening includes at least a first opening, a second opening, and a third opening.

16. The method of claim 15, wherein the aperture extends from a first exterior lateral edge of the first opening to a second exterior lateral edge of the second opening.

17. The method of claim 10, wherein the implantable device comprises a stent-graft having one or more stent components and one or more graft members.

18. An endoluminal device including:
an implantable device that is self-expanding and transitionable between a collapsed, delivery configuration, an intermediate deployment configuration having a greater diameter than the collapsed, delivery configuration, and an expanded, deployed configuration having a greater diameter than the collapsed, delivery configuration and the intermediate deployment configuration, the implantable device having a first end portion, a second end portion, and an intermediate portion located between the first and second end portions, the intermediate portion having a plurality of openings with a sidewall of the implantable device and providing access to an inner lumen of the implantable device, the plurality of openings including at least a first opening, a second opening, and a third opening; and
a constraining sleeve removably disposed around the implantable device in the collapsed, delivery configuration and the intermediate deployment configuration, the constraining sleeve having a single aperture aligning with the plurality of openings such that the single aperture provides access from an exterior to the inner lumen in the collapsed, delivery configuration and the intermediate deployment configuration, wherein the single aperture extends from a first exterior lateral edge of the first opening to a second exterior lateral edge of the second opening when the implantable device is in the intermediate deployment configuration, wherein partially deploying the constraining sleeve from the implantable device is operable to transition the implantable device from the collapsed, delivery configuration to the intermediate deployment configuration, and entirely deploying the constraining sleeve from the implantable device is operable to transition the implantable device from the intermediate deployment configuration to the expanded, deployed configuration.

19. The endoluminal device of claim 18, wherein each of the plurality of openings defines a retrograde portal when the endoluminal device is disposed in an antegrade position in a vasculature such that the retrograde portal is configured to provide access in a retrograde direction with respect to the antegrade position of the endoluminal device within the vasculature.

20. The endoluminal device of claim 18, wherein the implantable device comprises a stent-graft having one or more stent components and one or more graft members.

21. A method of treatment comprising:
delivering an endoluminal device in a vasculature, the endoluminal device comprising an implantable device, a secondary constraining sleeve disposed around the implantable device, and a primary constraining sleeve disposed around the secondary constraining sleeve such that the implantable device is in a collapsed, delivery configuration;
partially deploying the implantable device by removing the primary constraining sleeve from the secondary constraining sleeve, wherein, in a partially deployed configuration, an intermediate portion of the implantable device located between a first end portion and a second end portion of the implantable device expands through an aperture of the secondary constraining sleeve and beyond an outer surface of the secondary constraining sleeve, the intermediate portion having at least one opening in a sidewall providing access to an inner lumen of the implantable device, and the aperture aligning with at least a portion of the at least one opening with the sidewall, the aperture providing access from an exterior to the inner lumen in the partially deployed configuration while the secondary constraining sleeve restrains at least a portion of the implantable device in the partially deployed configuration; and
fully deploying the implantable device by removing the secondary constraining sleeve from the implantable device, wherein, in an expanded, deployed configuration, the implantable device has a greater cross-sectional distance at the first end portion and at the second end portion than at the intermediate portion.
